(19) United States
(12) Patent Application Publication
(10) Pub. No.: US 2009/0281422 A1
(43) Pub. Date: Nov. 12, 2009

(54) Multi-modality system for imaging in dense compressive media and method of use thereof

(76) Inventors: Khaled N. Salama, Troy, NY (US); Aly M. Ismail, Irvine, CA (US)

Correspondence Address: LAW OFFICES OF ROBERT HART, 28 EAST JACKSON BUILDING, 10TH FLOOR, SUITE H528, CHICAGO, IL, 60604, US

(21) Appl. No.: 12/151,355

(22) Filed: May 6, 2008

(51) Int. Cl. A61B 6/00; A61B 5/00

(52) U.S. Cl. 600/430; 600/437

(57) ABSTRACT

A multi-modality system and method for performing detection, characterization and imaging of materials and objects in dense compressive media, such as in medical soft tissue applications, is disclosed. Medical tissue applications include but are not limited to the detection and diagnosis of breast tumors. Generally, an ultrasound subsystem is employed to excite a region in the dense compressive media and a microwave subsystem is employed to collect detection, characterization and imaging information from the excited region. In one preferred embodiment, multiple focused oscillating high-frequency ultrasound wave beams are transmitted into the media. The resultant low beat-frequency wave creates a force inducing motion in the materials and objects in the media. A radio-frequency microwave subsystem detects that motion and produces images based upon the Doppler effects of the excited materials and objects.

### CROSS REFERENCE TO RELATED APPLICATIONS

Not applicable

### STATEMENT REGARDING FEDERALLY SPONSORED RESEARCH OR DEVELOPMENT

Not applicable

### INCORPORATION-BY-REFERENCE OF MATERIAL SUBMITTED ON A COMPACT DISC

Not applicable

### BACKGROUND OF THE INVENTION

1. Field of Invention

The present invention relates generally to the field of imaging in dense compressive media, and more particularly to a novel apparatus and method of use thereof for imaging in medical soft tissue applications such as orthopedics, dermatology, breast tumor scanning/detection and diagnosis/characterization. For simplicity of discussion, while applications are to be found in a wide range of medical and non-medical applications, this specification primarily addresses the exemplary application of breast tumor detection and diagnosis.

According to the U.S. National Library of Medicine and the National Institutes of Health, one in eight women will be diagnosed with breast cancer. One in sixteen women will die prematurely due to breast cancer. Breast cancer is more easily treated and often curable if it is discovered early. Breast cancer stages range from 0 to IV. The higher the stage number, the more advanced the cancer. According to the American Cancer Society (ACS), the 5-year survival rates for persons with breast cancer that is appropriately treated are as follows: 100% for Stage 0, 100% for Stage I, 92% for Stage IIA, 81% for Stage IIB, 67% for Stage IIIA, 54% for Stage IIIB, and 20% for Stage IV. Clearly, early detection is the primary factor in the successful treatment of breast cancer. Early breast cancer usually does not cause symptoms, therefore accentuating the importance of early detection devices and methods.

2. Discussion of Prior Art

The usefulness of methods and/or devices to perform breast cancer detection is well recognized. A variety of prior art methods and/or devices are directed to the problem. However, each prior art method and/or device possesses significant disadvantages.

The principal methods of detecting breast cancer are clinical physical examination, self-examination, and X-ray mammography. Efforts have been made to develop alternative solutions to the problem of breast cancer detection, including magnetic resonance imaging (MRI) and microwave radar imaging.

In a clinical physical examination, a doctor performs a tactile physical examination of the breasts, armpits, and the neck and chest area. The physical examination is intended to discover lumps indicative of cancer. However, the clinical physical examination cannot identify the nature of the lump and lacks the sensitivity or resolution of other methods.

The breast self-examination is essentially the same as the clinical physical examination, but it is performed by the subject outside of the clinical environment. The breast self-examination is similar in benefit and limitation to the clinical physical examination.

X-ray mammography is currently the only FDA-certified early breast cancer screening technology. X-ray mammography, in some cases, can detect breast cancers before they can be detected by a physical examination. In either a sitting or a standing position, one breast at a time is rested on a flat surface that contains an X-ray exposure plate. A device called a compressor is pressed firmly against the breast to flatten out the breast tissue. This results in substantial discomfort to the patient. The patient holds her breath as a series of X-ray pictures are taken from several angles. Deodorant, perfume, powders and jewelry must be removed to prevent blockage of the X-rays. In each examination, the patient is exposed to destructive ionizing radiation, thus incurring a risk of realizing an induced breast tumor. X-ray mammography is considered a health risk for women who are pregnant or breast-feeding, and it is not recommended for women under the age of fifty. Further, X-ray mammography is a very poor method for early-stage cancer detection. In a recent study, only 52 percent of high-grade ductal carcinoma in situ (DCIS), the form most likely to develop into invasive cancer, were detected by X-ray mammography. “MRI for Diagnosis of Pure Ductal Carcinoma In Situ: A Prospective Observational Study,” Christiane Kuhl, et. al., The Lancet, vol. 370, issue 9586, 11 Aug. 2007, pages 485-492.

Due to the shortcomings in these current methods, there exists an on-going search for other effective methodologies. Magnetic resonance imaging (MRI) and microwave radar are two solutions of interest.

Magnetic resonance imaging (MRI) employs powerful magnets and radio waves to generate images inside the body. The magnetic field produced by an MRI is about ten thousand times greater than the Earth's magnetic field. The magnetic field polarizes the magnetic moment of hydrogen atoms in the body. When properly tuned radio waves are then transmitted through the body, they are absorbed in different ways depending on the types of tissue they encounter. The resulting radio signal can thus often distinguish healthy versus cancerous tissue. MRI represents a substantial improvement over X-ray mammography in terms of early detection, detecting 98 percent of high-grade DCIS compared with 52 percent detection by X-ray mammography. “MRI for Diagnosis of Pure Ductal Carcinoma In Situ: A Prospective Observational Study,” Christiane Kuhl, et. al., The Lancet, vol. 370, issue 9586, 11 Aug. 2007, pages 485-492.

While MRI offers improved detection over X-ray mammography and eliminates the risk associated with ionizing radiation, it brings other attendant problems. Many patients find the MRI procedure uncomfortable. The patient may be required to fast from four to six hours prior to the scan. Then, the patient lies on a narrow table which slides into the middle of the MRI scanner. The MRI machine may induce anxiety in patients with a fear of confined spaces. Further, the MRI machine produces loud percussive and buzzing noises which may be disconcerting to the patient. Finally, because several sets of images are required, each taking from two to fifteen minutes, the patient must be exposed to the MRI environment for an hour or longer. The patient is required to lie motionless for this long period of time because excessive movement can blur MRI images and cause errors. In addition, because the magnet is very strong, certain types of metal can cause significant errors in the images, and the strong magnetic fields created during an MRI can interfere with certain medical implants. Persons with pacemakers or other metallic objects in the body, such as ear implants, brain aneurism clips, artificial heart valves, vascular stents and artificial joints should not be exposed to MRI. Patients have been harmed in MRI machines when they did not remove metal objects from their clothes or when metal objects were left in the room by others. Finally, the high cost of procuring and operating an MRI machine, and the lack of people skilled at reading breast MRIs, suggests it will not replace X-ray mammography as a routine screening methodology anytime soon.

More recently, research has turned to microwave radar techniques for soft tissue imaging. Radar imaging may provide detection capability that is superior to X-ray mammography, and equivalent to MRI, at a much lower implementation complexity and cost. Radar imaging offers a low-stress, low health risk solution, requiring short exposure periods without the dangers or discomforts associated with X-ray mammography or MRI. The scientific principles are defined and experimentally demonstrated in a publication entitled “Microwave Imaging via Space-Time Beamforming: Experimental Investigation of Tumor Detection in Multilayer Breast Phantoms,” Xu Li, et. al., IEEE Transactions on Microwave Theory and Techniques, vol. 52, no. 8, August 2004. These authors utilize the 1 to 11 GHz frequency range to demonstrate the engineering tradeoffs of superior spatial resolution at higher frequencies versus deeper tissue penetration at lower frequencies. They demonstrate the effectiveness of radar imaging principles in breast tumor detection applications by employing a two-dimensional scanning methodology to synthesize a two dimensional antenna array.

Two principal radar methodologies are found in the art: pulse-delay radar and frequency modulation (FM) ranging radar. In the pulse-delay radar method, a single frequency, short-duration wave is transmitted into the breast. The time between transmission and the detection of the returned scattered wave is measured to enable calculation of the tumor's position within the breast. In the FM ranging method, also referred to as chirping, the frequency of the transmitted wave is varied over a period of time. Then, the frequency of the reflected radar wave is compared against the transmitted wave, enabling the round-trip distance of the signal to be calculated, and thus the location of the reflecting tumor. Each of these methods rely on the fact that a region of cancerous tissue within a breast will strongly reflect microwave energy and thus provide strong contrast within a matrix of surrounding normal tissue.

Wang, U.S. Pat. No. 6,041,248 discloses a method for ultrasound modulated optical tomography of dense turbid media. In this method, an ultrasound wave is transmitted into a turbid medium. Coherent light from a laser is passed through the medium where it is modulated by the ultrasound wave. Light passing through the turbid medium is detected, and differences in light intensity at different frequencies are used to determine the location of objects in the turbid medium. While this method demonstrates the usefulness of ultrasound excitation in imaging applications, the disclosed laser-based method is invasive, and more applicable to detection than diagnosis.

Non-invasive detection and diagnosis methods utilizing acoustic means for both excitation of the tissues and for measurement and imaging of the excited tissues are known generally as vibro-acoustography and harmonic motion imaging. These methods utilize focused, oscillating high-frequency ultrasound input waves having slightly different frequencies to excite the target tissue at the intersection of the beams. These input waves propagate and interact producing a series of harmonic waves. One resultant harmonic is a low-frequency wave resulting from the cancellation of the high-frequency components of the input waves, generally known as the beat frequency. This low-frequency harmonic component produces a force that excites the target tissue. In vibro-acoustography, a hydrophone receives acoustic waves generated by the motion induced in the tissue and processes that received output into imaging information. Harmonic motion imaging utilizes the same ultrasonic tissue excitation scheme employed in vibro-acoustography, but incorporates an pulse-echo ultrasound transceiver to perform the measurement and imaging function. Hynyen, et. al., U.S. Pat. No. 6,984,209 discloses a harmonic motion imaging method which includes transmitting a first and second oscillating ultrasound energy beams from first and second sources into the object such that the beams intersect at the desired region to induce vibration of the desired region, transmitting pulsed ultrasound energy from a third source into the desired region, receiving signals from the desired region due to the echo of energy from the third source, and analyzing at least one of amplitude, phase and frequency of the vibration of the desired region indicated by the received signals to determine the property of the desired region.

Vibro-acoustography and harmonic motion imaging methods both take advantage of low-frequency ultrasound harmonic components from multiple high-frequency ultrasound wave inputs to excite the target tissue, and rely on acoustic methods for measuring and imaging the excited tissues. Acoustic methods of measurement and imaging suffer limitations in terms of noise, contrast and speckle. Microwave detection methods offer a factor of five improvement in detection sensitivity and diagnosis capacity over ultrasound methods. Ultrasound methods rely on the measurement of variations in the mechanical properties of benign tissue and cancerous tumors, which are not large. On the other hand, microwave methods take advantage of the difference in dielectric constants associated with the water content of benign tissue and cancerous tumors, which vary dramatically.

### BRIEF SUMMARY AND OBJECTS OF THE INVENTION

In view of the foregoing disadvantages inherent in the known devices and methods in the prior art, the present invention provides a novel multi-modality system and method for performing detection, characterization and imaging of materials and objects in dense compressive media, particularly but not exclusively in medical soft tissue applications. Specifically, the present invention involves coupling an ultrasound subsystem for exciting target tissues with a microwave subsystem for measuring the response and imaging the target tissues. The present invention combines the superior penetration and resolution characteristics of focused high-frequency ultrasound input waves, the superior excitation capability of resultant low-frequency ultrasound harmonics, and the superior penetration and detection capacity of microwave detection and imaging.

The present invention combines the superior penetration and resolution characteristics of the ultrasound excitation modality with the high-contrast capability of the microwave imaging modality. A further embodiment of the present invention utilizes the ultrasound excitation modality and combines acoustic and microwave measurement and imaging modalities.

It is the primary object of the present invention to enhance early detection and diagnosis capability using a low-cost and minimally uncomfortable imaging modality.

It is an object of an alternative embodiment of the present invention to avoid the complexity and cost associated with mechanical scanning by employing an ultrasound transducer array in place of scanning ultrasound microwave transducers.

Another object of the present invention is to enable application of low-cost components, such as compact radio frequency components developed for the wireless communications industry and existing ultrasound components.

A further object of the present invention is to achieve a small form factor, relative to MRI and X-ray devices, to reduce cost, enhance flexibility and convenience, and enable design of a single handheld device.

It is an object of the present invention to enable three-dimensional detection and diagnosis imaging. In one alternative embodiment of the present invention, ultrasound and microwave subsystem combinations are implemented in multiple axes. These multi-axis subsystems cooperate to provide superior three-dimensional imaging capability. In another embodiment of the present invention, ultrasound arrays may be used to develop three-dimensional maps of the target breast.

It is an object of the present invention to minimize patient discomfort. The present invention enables a short imaging time while eliminating the discomfort of X-ray mammography breast compression and confining MRI apparatuses, and the stress associated with ionizing radiation exposure. The present invention requires merely soft compression to maintain contact between the target breast and the ultrasound transducers and microwave antenna.

It is a further object of the present invention to eliminate health risks to the patient. The present invention eliminates the risk of short-term or long-term deleterious affects associated with ionizing radiation exposure in X-ray mammography, and the risks associated with exposure to powerful magnetic fields in MRI.

Other advantages of the present invention may become readily apparent to those with skill in the art from the following figures, descriptions and claims. As will be appreciated by those of skill in the art, the present invention may be embodied as systems or methods.

### BRIEF DESCRIPTION OF THE DRAWINGS

The exact nature of this invention, as well as all its objects and advantages, will become readily apparent and understood upon reference to the following detailed description when considered in conjunction with the accompanying drawings, in which like reference numerals designate like parts throughout the figures thereof, and wherein:

FIG. 1 shows the orientation of the system with respect to the patient and the imaging target breast in one preferred embodiment of the present invention.

FIG. 2 provides a schematic representation of the ultrasound subsystem.

FIG. 3 provides a schematic representation of the microwave imaging subsystem.

FIG. 4 shows the ultrasound wave transmission through the subject breast, the resultant displacement of the target tumor, and the display of the reflected microwaves resulting from the ultrasound excitation of the tumor.

FIG. 5 shows an alternative embodiment of wherein the microwave antenna is oriented on the same side of the breast as are the ultrasound transducer.

FIG. 6 shows an alternative embodiment of the ultrasound wave transmission transducers featuring a confocal ultrasound transducer configuration.

FIG. 7 illustrates an alternative embodiment wherein an ultrasound modality is employed in combination with a microwave modality to perform the detection and imaging functions.

FIG. 8 illustrates an alternative embodiment wherein an acoustic modality (hydrophone) is employed in combination with a microwave modality to perform the detection and imaging functions.

FIG. 9 illustrates an alternative embodiment wherein an acoustic modality (hydrophone) is employed in combination with a microwave modality to perform the detection and imaging functions, and a confocal ultrasound transducer performs the excitation function.

FIG. 10 shows an alternative embodiment of the ultrasound subsystem featuring the use of ultrasound arrays.

FIG. 11 shows an alternative embodiment of the ultrasound subsystem in which a single ultrasound transducer is employed to input integrated ultrasound waves into the target breast.

### DETAILED DESCRIPTION OF THE PREFERRED EMBODIMENTS

The following description is provided to enable any person skilled in the art to make and use the invention and sets forth the best modes contemplated by the inventor of carrying out his invention.

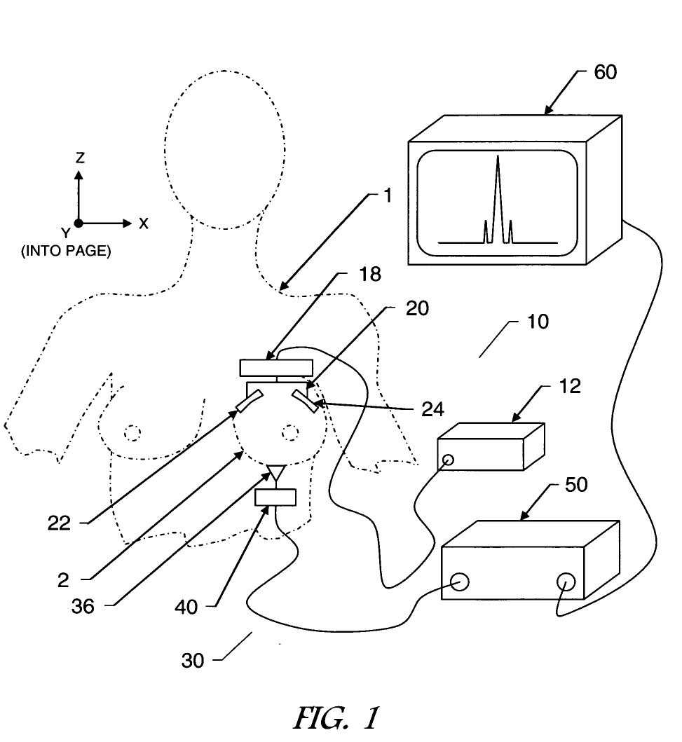

Referring now to the drawings, FIG. 1 shows the orientation of the system with respect to the patient 1 and the imaging target breast 2 in one preferred embodiment of the present invention. An ultrasound subsystem 10 and a microwave imaging subsystem 30 are employed in combination to detect and diagnose tumors in the breast 2. A first ultrasound transducer 22, a second ultrasound transducer 24 and a microwave antenna 36 are oriented with respect to the target breast 2 of the patient 1. A radio frequency transceiver 40 generates and transmits microwave signals to the microwave antenna 36. The microwave antenna 36 transmits microwaves into the target breast 2. Reflected microwaves are collected by the microwave antenna 36 and received by the radio frequency transceiver 40. A computer/signal and data processor 50 containing signal processing circuitry and data processing algorithms processes the output of the radio frequency transceiver 40 and sends the resultant data to the display 60 for access by the technician. The display may optionally be an oscilloscope or a spectrum analyzer. The data may be usefully represented as individual spectra, one-dimensional line scans, two-dimensional cross-sectional constructions, or volume images. A scan controller/actuator 18 working in combination with a mechanical actuator 20 orients ultrasound transducers 22/24 to enable scanning of the entire target breast 2. An ultrasound electronics assembly 12 generates and transmits electronic ultrasound waveform signals to the ultrasound transducers 22/24. The ultrasound transducers 22/24 transmit ultrasound waves to the target breast 2 to excite the tissues therein.

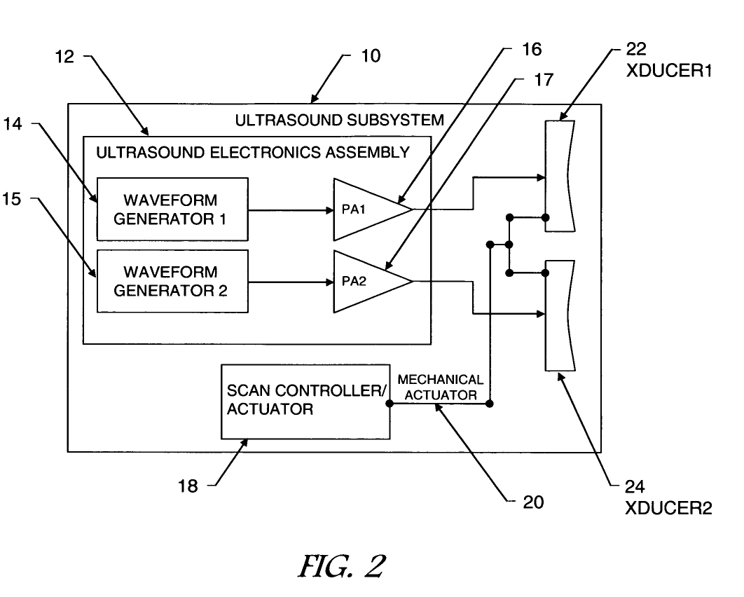

FIG. 2 provides a schematic representation of the ultrasound subsystem 10. An ultrasound electronics assembly 12 is shown housing a first waveform generator 14 and a second waveform generator 15, and a first power amplifier 16 and a second power amplifier 17. Waveform generator 14 produces an input ultrasound waveform 8 having frequency ƒ₁. Waveform generator 15 produces an input ultrasound waveform 9 having frequency ƒ₂. Power amplifier 16 conditions input ultrasound waveform 8 and transmits ultrasound waveform 8 to ultrasound transducer 22. Power amplifier 17 conditions input ultrasound waveform 9 and transmits ultrasound waveform 9 to ultrasound transducer 24. Ultrasound transducer 22 transmits the amplified input ultrasound wave 8 into the target breast 2. Ultrasound transducer 24 transmits the amplified input ultrasound wave 9 into the target breast 2. To maximize transmission of the ultrasound waves 8/9 into the target breast 2, an ultrasound conductive gel may be used at the interface of the ultrasound transducers 22/24 and the target breast 2. In a preferred embodiment of the present invention, the ultrasound transducers 22/24 must be physically relocated to perform a scan of the entire breast 2. This scanning function is performed by a scan controller/actuator 18 working in combination with a mechanical actuator 20.

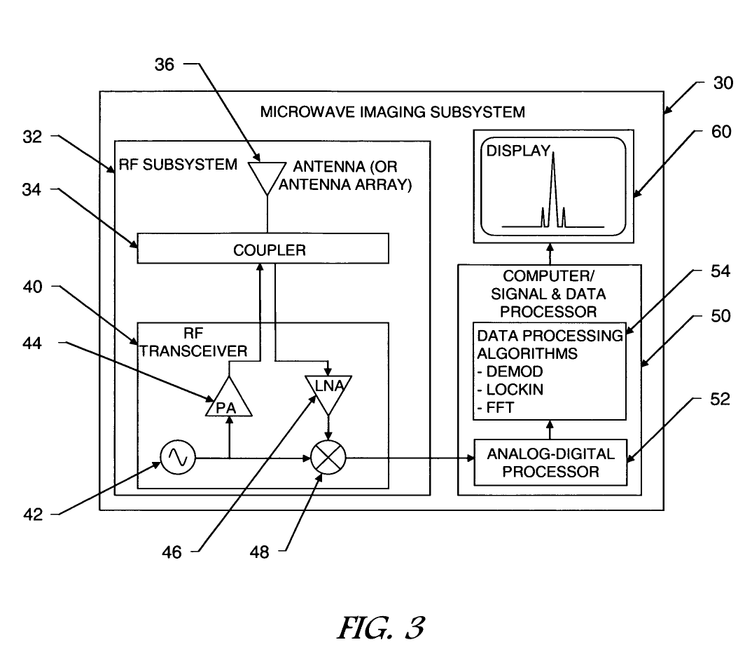

FIG. 3 provides a schematic representation of the microwave imaging subsystem 30 comprising an RF subsystem 32, a computer/signal & data processor 50 and a display 60. The RF subsystem 32 comprises a microwave antenna 36, a coupler 34, and an RF transceiver 40. The RF transceiver 40 comprises a waveform generator 41, a power amplifier 44, a linear noise amplifier 46 and a mixer 48. The waveform generator 42 produces an input microwave 6. The power amplifier 44 conditions the input microwave 6 and transmits said microwave 6 through the RF coupler 34 to the RF antenna 36. The microwave antenna 36 transmits the microwave 6 into the target breast 2. To efficiently transmit the microwave 6 to the breast 2, the RF antenna 36 is in physical contact with the breast 2. In a preferred embodiment of the present invention, the RF antenna 36 is made from a material that closely matches the dielectric constant of the breast 2. In an alternative embodiment, a dielectrically loaded antenna, in which the antenna 36 is embedded in a material that matches the dielectric constant of the breast 2, may be employed to reduce reflections. Due to the wide propagation angle of the microwave 6 in the breast 2, it is not necessary to move the RF antenna to scan the breast 2. However, an alternative embodiment of the present invention may employ a microwave antenna 36 scanning means, if desired. Microwaves reflected by normal/cancerous tissue boundaries and/or inclusions are collected by the microwave antenna 36 and transmitted through the coupler 34 to a linear noise amplifier 46. Input microwaves from the waveform generator 42 and reflected microwaves from the linear noise amplifier 46 are passed through a mixer 48 and conveyed to an analog-digital processor 52. Data processing algorithms 54 such as demodulation, and lockin detection or fast Fourier transform algorithms operate on the digital data from the analog-digital processor 52. The resultant frequency and power data is transmitted to a display 60 for viewing by the technician.

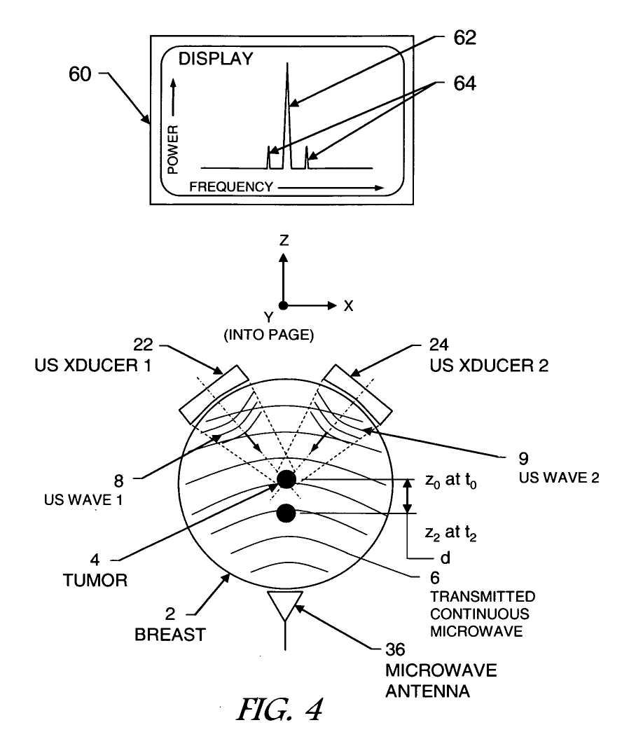

FIG. 4 shows transmission of microwaves 6 and of the first and second ultrasound waves 8/9 into the subject breast, the resultant displacement d of the target tumor 4, and the display 60 of the spectral representation of the microwaves reflected from the excited tumor 4.

At time t₀, the unexcited tumor 4 is at rest in location z₀ and the microwave antenna 36 is transmitting microwaves into the breast 2. In one preferred embodiment of the present invention, a continuous microwave is employed. It is anticipated that other input waveforms and methods, such as frequency modulation and pulse-delay, can eventually be used to reduce clutter signals and improve the probability of tumor detection. Prior to activation of the ultrasound transducers 22/24, microwaves are reflected back to the microwave antenna 36 from the internal boundaries of the breast and from inclusions in the breast 2 such as a tumor 4. The reflected microwaves are of the same frequency as the transmitted input microwaves 6. The reflected microwave appears on the display 60 as a power spike 62 at the frequency of the transmitted wave. No position or shape information of the tumor 4 is detectable prior to activation of the ultrasound transducers 8/9.

At time t₁, a first ultrasound transducer 22 transmits a first ultrasound wave 8 having a frequency ƒ₁ into the breast 2 and a second ultrasound transducer 24 transmits a second ultrasound wave 9 having a frequency ƒ₂ into the breast 2. The lenses of the ultrasound transducers 22/24 are designed to create focused ultrasound beams which intersect at the target tumor 2. In the preferred embodiment, ultrasound frequencies ƒ₁ and ƒ₂ are high frequencies with a small differential, or beat frequency (ƒ₁−ƒ₂). The high frequencies of the input ultrasound waves 8/9 provide superior resolution and focus capability, but poor tissue displacement force. But as the first and second high-frequency ultrasound waves propagate and interact, they produce a series of harmonic waves. One resultant harmonic is a low-frequency wave at the beat frequency (ƒ₁−ƒ₂) resulting from the cancellation of the high-frequency components of the input waves. This low-frequency harmonic component produces a force that excites and displaces the target tissue and tumor 4. Due to the non-linear density and elastic properties of tissues and tumors in the breast, the displacement of target tumor 4 can be detected. Expressed mathematically:

Source₁=cos(2πƒ₁t)=cos(ω₁t)

Source₂=cos(2πƒ₂t)=cos(ω₂t)
Where ω=2πƒ=angular frequency, and t=time
   Due to high power at the intersection point of the ultrasound beams, non-linearity effects of the tissue become pronounced and the mixing of the two ultrasound signals becomes:

$$\begin{matrix}Resultant = {a}_{1} [ cos ( {ω}_{1} t ) + cos ( {ω}_{2} t ) ] + {{a}_{2} [ cos ( {ω}_{1} t ) + cos ( {ω}_{2} t ) ]}^{2} + … \\ = {a}_{1} cos ( {ω}_{1} t ) + {a}_{1} cos ( {ω}_{2} t ) + {a}_{2} {cos}^{2} ( {ω}_{1} t ) + {a}_{2} {cos}^{2} ( {ω}_{2} t ) + \\ 2 {a}_{2} cos ( {ω}_{1} t ) cos ( {ω}_{2} t ) + … \\ = {a}_{1} cos ( {ω}_{1} t ) + {a}_{1} cos ( {ω}_{2} t ) + {a}_{2} [ 0.5 cos ( 2 {ω}_{1} t ) + 0.5 ] + \\ {a}_{2} [ 0.5 cos ( 2 {ω}_{2} t ) + 0.5 ] + \\ {a}_{2} [ cos ( ( {ω}_{1} + {ω}_{2} ) t ) ) + cos ( ( {ω}_{1} - {ω}_{2} ) t ) ] + …\end{matrix}$$

Where a=a constant coefficient dependent upon the non-linearity of the tissue
   The resultant displacement d of the tissue is given by the equation:

d=1/(2π=ƒ)*sqrt(2FZ)
Where
   F=energy flux (i.e., power per area),
   Z=tissue acoustic impedance, typically ˜1.5e⁶ kg/m²/s, and
   ƒ=acoustic frequency (in this case ƒ₁−ƒ₂).
      Since ω₁ and ω₂ are high frequency to achieve good resolution, then terms with twice the frequency (cos(2ω₁), cos(2ω₁) and cos(ω₁+ω₂)) will be of high frequency and their effect on the motion will be limited. On the other hand, if ω₁ and ω₂ are selected to be close to each other such that (ω₁−ω₂) would be very small (i.e., in order of 100s-1000s Hz), then the term cos((ω₁−ω₂)t) will lead to a large displacement.

At time t₂, the low-frequency ultrasound component impacts the tumor 4 and displaces the tumor 4 to location Z₂. As the low-frequency ultrasound wave passes the tumor 4, the tumor oscillates between location Z₂ and z₀ before coming to rest again at essentially the initial location z₀. The ultrasound wave travels at a significantly lower rate of speed than the microwave 6. As the tumor 4 oscillates between position z₀ and position Z₂, the Doppler effect results in a shift in the frequency of the reflected microwave. These frequency shifts appear on the display 60 as frequency sidebands 64. Presence of these sidebands indicates the presence of a tumor 4. The sidebands 64 are short lived, essentially lasting for the duration of the ultrasound pulse passing through the tumor 4.

The power of the sidebands 64 is determined through displacement analysis. If a signal is reflected off of a target whose range is changing with time according to r(t)=r₀+Δr(t), the received signal can be written as:

s(t)=cos [ω_{c}t+2π−Δr(t)/λ+φ₀]

Where ω_{c} is the carrier frequency and φ₀ is the phase

For a small-amplitude oscillation of a target with a displacement d and a modulation frequency fm, the range is given by:

Δr(t)=d sin(ωₘt)

And thus the signal becomes

s(t)=cos [ω_{c}t+2π−(d/λ)sin(ωₘt)+φ₀]

For d<<λ, this expression is simply the narrowband FM situation:

$$\begin{matrix}f ( t ) = cos [ {ω}_{c} t + ( d / λ ) sin ( {ω}_{m} t ) ] \\ = cos ( {ω}_{c} t ) cos ( ( d / λ ) sin ( {ω}_{m} t ) ) - sin ( {ω}_{c} t ) sin ( ( d / λ ) sin ( {ω}_{m} t ) ) \\ = cos ( {ω}_{c} t ) - ( d / 2 λ ) [ cos ( {ω}_{c} t - {ω}_{m} t ) - cos ( {ω}_{c} t + {ω}_{m} t ) ]\end{matrix}$$

Each sideband is smaller than the carrier by:

P_{sideband}=10 log(d²/4λ²)=20 log(πf_{c}d/c)dBc.

Radio frequency sensitivity is determined by the equation:

Sensitivity=NF+KT+10 log(BW)+SNR−10 log(Average)

### Where

NF: The receiver input referred noise figure (Typically 3-5 dB)

KT: Thermal noise power density (−174 dBm/Hz)

BW: Receiver noise bandwidth in Hz (typically 1-2 MHz)

SNR: Required detector SNR in dB (20 dB)

Average: Coherently collected samples over sample time

If sensitivity is not sufficient, and to give system sensitivity a boost, a continuous wave may be employed such that:

Sensitivity=NF+KT+10 log(BW)+SNR−10 log(Average)−10 log(gain)

### Where

gain:gain achieved due to applying continuous wave

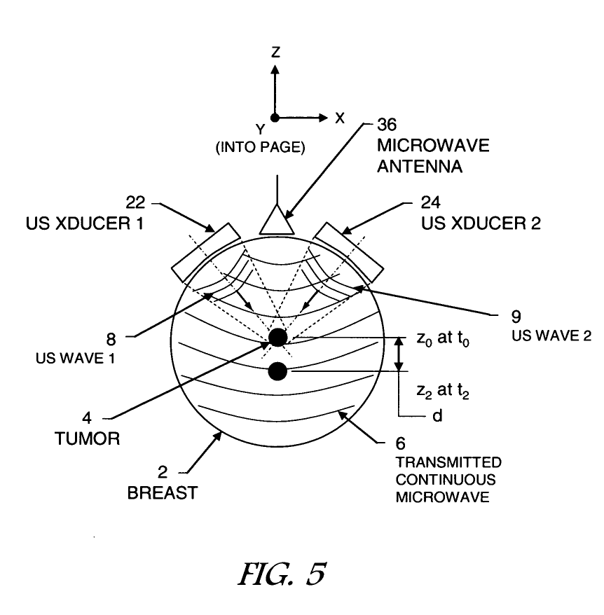

FIG. 5 shows an alternative embodiment of wherein the microwave antenna 36 is oriented on the same side of the breast as the ultrasound transducers 22/24. The concept of operation and the method of use are identical to that of the embodiment of FIG. 4, but may provide packaging advantages over that embodiment, such as enabling design of a single handheld device.

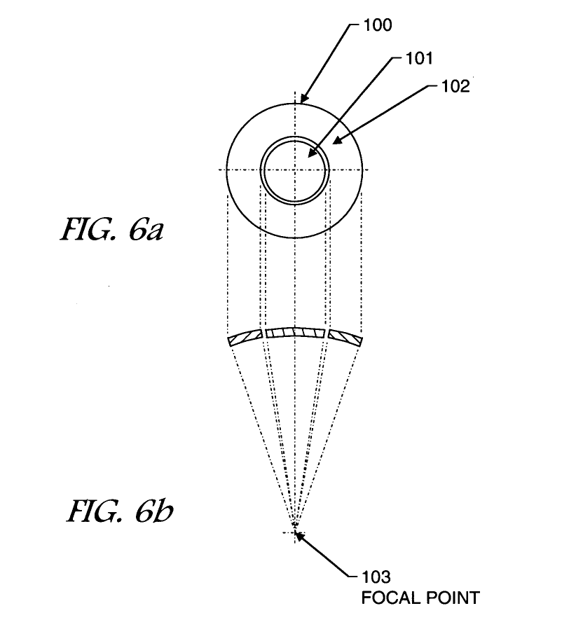

FIG. 6 shows an alternative embodiment of the ultrasound wave transmission transducers featuring a confocal ultrasound transducer 100 configuration. FIG. 6a presents a plan view of the confocal ultrasound transducer 100. FIG. 6b presents a cross-sectional view of the confocal ultrasound transducer 100. In this embodiment, the first ultrasound transducer 101 and second ultrasound transducer 102 are implemented in a fixed physical relationship to one another, with both ultrasound transducers 101/102 focused an a single focal point 103 as illustrated in FIG. 6b. This confocal configuration scheme may be employed to package any number of transmission and/or reception ultrasound transducer elements.

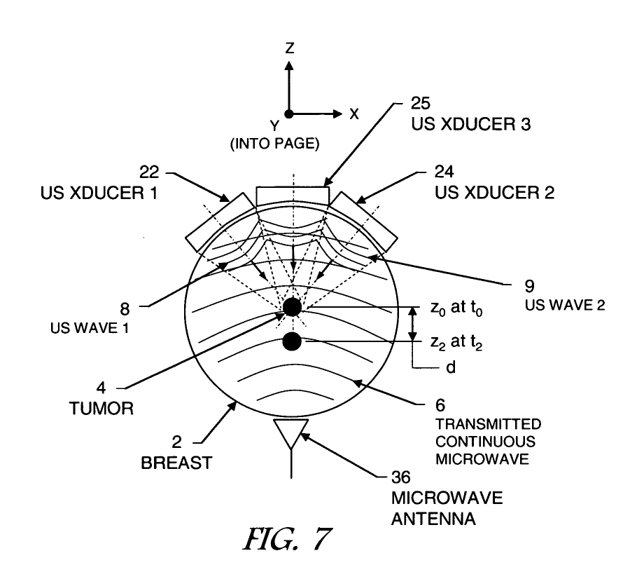

FIG. 7 illustrates an alternative embodiment wherein an ultrasound modality is employed in combination with a microwave modality to perform the detection and imaging functions. In this embodiment, a third ultrasound transducer 25 is incorporated into the configuration shown in FIG. 4. Ultrasound transducer 25 augments the detection and imaging function performed by microwave antenna 36. Ultrasound transducer 25 transmits a third focused ultrasound beam into the target region. Echo signals indicative of reflected energy from said third focused ultrasound beam are received and analyzed to determine the property of the target region.

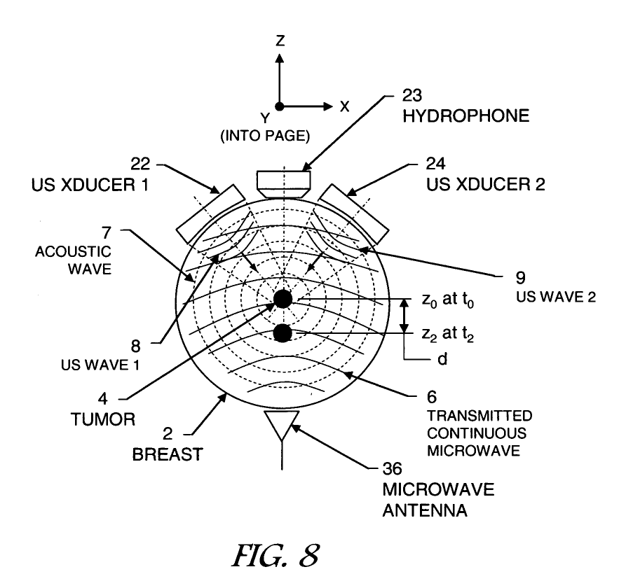

FIG. 8 illustrates an alternative embodiment wherein an acoustic modality is employed in combination with a microwave modality to perform the detection and imaging functions. In this embodiment, an acoustic hydrophone 23 is incorporated into the configuration shown in FIG. 4. Acoustic hydrophone 23 augments the detection and imaging function performed by microwave antenna 36. The acoustic hydrophone 23 receives acoustic waves 7 generated by the motion induced in the tissue. A signal processor transforms the detected acoustic input into imaging information.

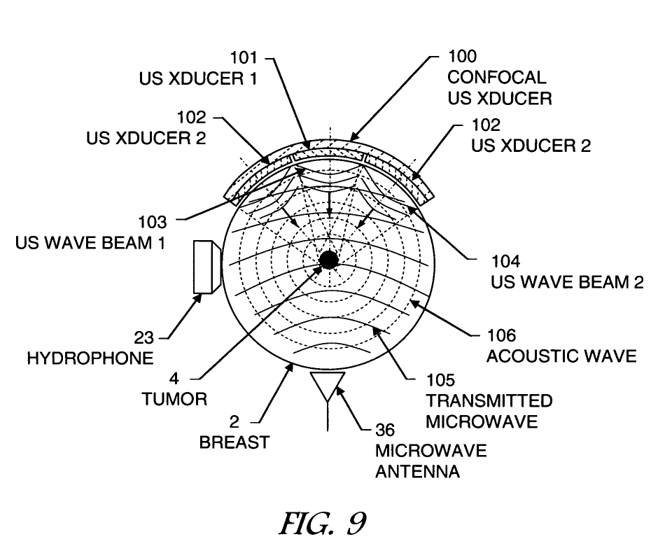

FIG. 9 presents an alternative embodiment of the present invention wherein the exemplary confocal ultrasound transducer 100 of FIG. 6 is used in place of the individual ultrasound transducers 22/24 shown in FIG. 7.

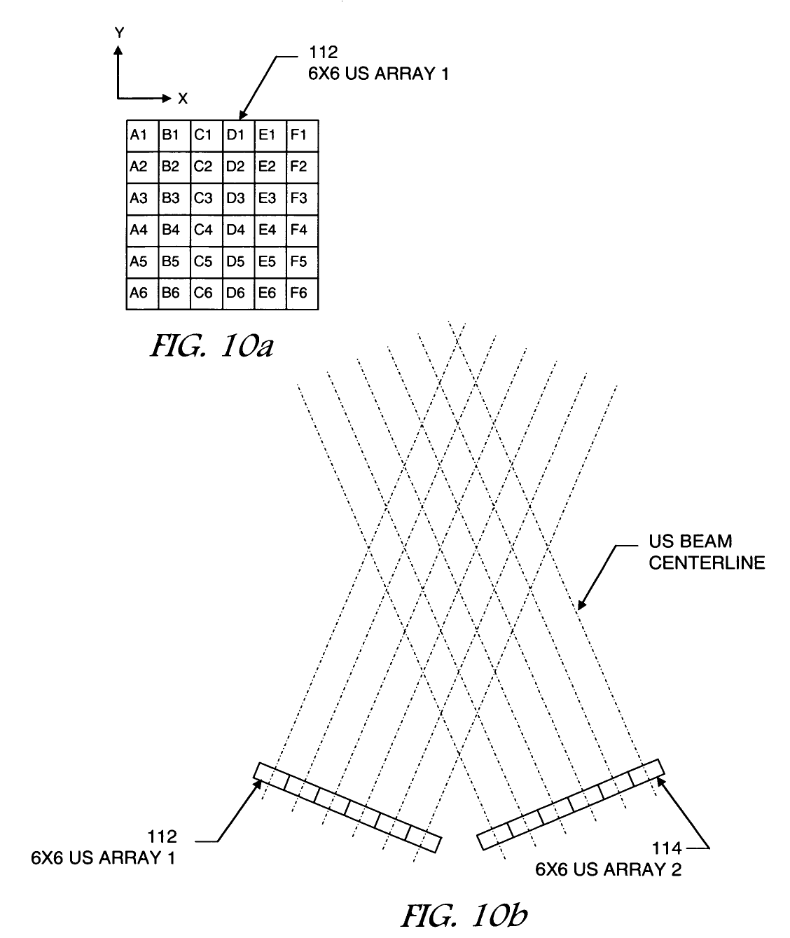

An alternative embodiment of the present invention employs ultrasound arrays 112/114 in place of the single scanning ultrasound transducers 22/24. FIG. 10 illustrates an exemplary ultrasound array implementation. FIG. 10a presents a plan view of a 6×6 ultrasound array 112 with ultrasound transducer element arranged in a matrix of rows A through B along the x-axis and columns 1 through 2 along the y-axis. FIG. 10b presents a side view of two 6×6 ultrasound arrays 112/114. The two ultrasound arrays operate in cooperation to transmit ultrasound waves into the breast. By selectively activating ultrasound elements of each of the arrays, the paired arrays 112/114 can focus input ultrasound energy waves at the intersection of the ultrasound beam centerlines of the activated ultrasound elements. Further, electronic tuning of the dual-array system permits focus between the centerline intersection points. This embodiment permits detection to be performed throughout a large volume of the breast without the need for scanning. This embodiment trades the physical complexity and longer examination times associated with scanning implementations for the greater electronic implementation complexity of the ultrasound array implementation. While a symmetrical 6×6 array is shown to illustrate the concept, many array configurations may be usefully employed. 3×120 and 5×120 non-symmetrical arrays and 1×120 linear arrays are found in literature.

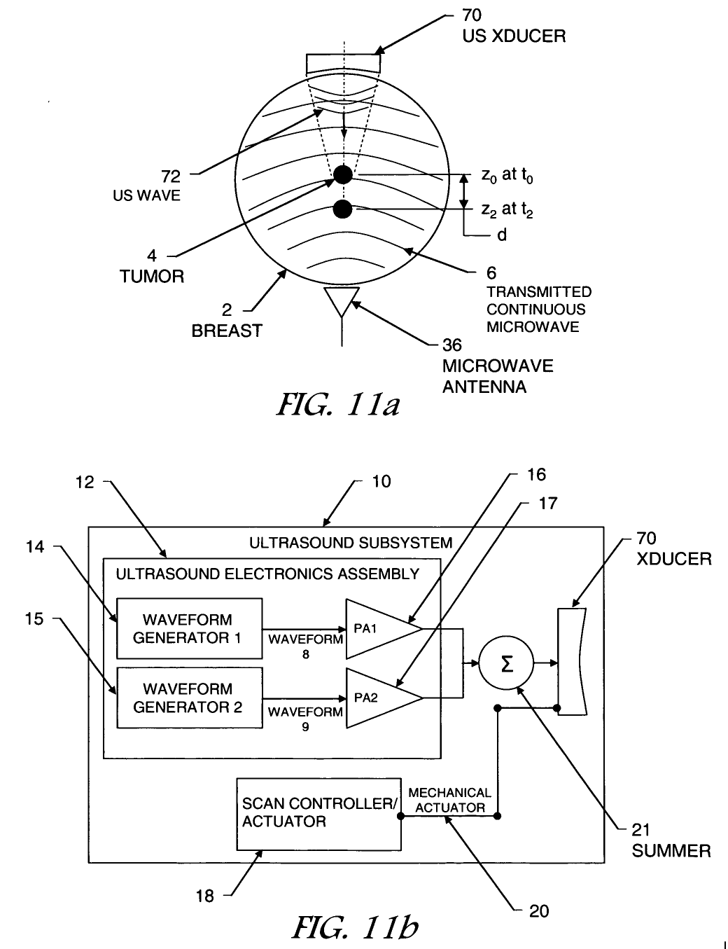

FIG. 11 shows an alternative embodiment of the ultrasound subsystem in which a single ultrasound transducer 70 is employed to input integrated ultrasound waves 72 into the target breast. FIG. 11a presents one preferred embodiment of this alternative wherein the microwave antenna 36 and the ultrasound transducer 70 are positioned on opposite sides of the breast 2. One alternative embodiment includes positioning a single annular ultrasound transducer around the microwave antenna 36 on the same side of the breast 2. FIG. 11b provides a schematic representation of the ultrasound system. In this embodiment, waveform generator 14 produces an input ultrasound waveform 8 having frequency ƒ₁. Waveform generator 15 produces an input ultrasound waveform 9 having frequency ƒ₂. Power amplifier 16 conditions input ultrasound waveform 8 and transmits ultrasound waveform 8 to a summer 21. Power amplifier 17 conditions input ultrasound waveform 9 and transmits ultrasound waveform 9 to the summer 21. The summer 21 combines the input ultrasound waveforms 8/9 and transmits the combined waveforms 8/9 to a single ultrasound transducer 70. The ultrasound transducer 70 transmits the multiple input ultrasound waves 8/9 in a single, integrated focused ultrasound beam 72, comprising both ƒ₁ and ƒ₂ components, into the breast 2. As discussed relative to the embodiment of FIG. 4, as the combined high-frequency ultrasound waves 72 propagate and interact, they produce a series of harmonic waves. One resultant harmonic is a low-frequency wave at the beat frequency (ƒ₁−ƒ₂) resulting from the cancellation of the high-frequency components of the input waves. This low-frequency harmonic component produces a force that excites and displaces the target tissue and tumor 4. Due to the non-linear density and elastic properties of tissues and tumors in the breast, the displacement of target tumor 4 can be detected.

It is obvious and anticipated that various embodiments of the present invention may be exercised in ways other than illustrated in the Figures. Such alternative embodiments are within the contemplation of the present invention.

It is obvious and anticipated that the present invention may be adapted to a variety of applications in both medical and non-medical fields. The field of medical soft tissue imaging, includes orthopedics, dermatology, breast tumor detection and characterization, and other medical applications. Such alternative applications are within the contemplation of the present invention.

It is obvious and anticipated that the physical implementation of the present invention may be varied without departing from the spirit of the invention. Elements and components may be implemented, added, interchanged, combined and/or packaged in a variety of embodiments. Various changes may be effected in structure, design, choice of components and materials, etcetera without departing from the spirit of the present invention. Such alternative embodiments, elements and implementations are within the contemplation of the present invention.

Accordingly, the scope of the invention should be determined not by the embodiments illustrated, but by their legal equivalents.

The following references are of utility in understanding the foregoing specification:
Li, Xu, et. al. (2004): Microwave Imaging via Space-Time Beamforming: Experimental Investigation of Tumor Detection in Multilayer Breast Phantoms, IEEE Transactions on Microwave Theory and Techniques, Vol. 52, No. 8, pp 1856-1865.
Reinberg, Steven (Aug. 10, 2007): MRI Beats Mammograms at Spotting Early Breast Cancer, HealthDay News <http://www.healthday.com/Article.asp?AID=607199>.
Nanda, R. (2007): Breast Cancer, Medline Plus Medical Encyclopedia, the U.S. National Library of Medicine and the National Institute of Health <http://www.nlm.nih.gov/medlineplus/ency/article/000913.htm>.
A. Alizad, M. Fatemi, L. E. Wold and J. F. Greenleaf, “Performance of Vibro-Acoustography in Detecting Microcalcifications in Excised Human Breast Tissue: A Study of 74 Tissue Samples,” IEEE Trans. Med. Imaging., vol. 23, pp. 307-312, March 2004.
C. Maleke, J. Luo and E. E. Konofagou, “2D Simulation of the Harmonic Motion Imaging (HMI) With Experimental Validation,” IEEE Ultrasonics Symposium, pp. 797-800, 2007.
E. E. Konofagou, M. Ottensmeyer, S. L. Dawson and K. Hynynen, “Harmonic Motion Imaging—Applications in the Detection of Stiffer Masses,” IEEE Ultrasonics Symposium, pp. 558-561, 2003.

What is claimed is:

1. A system for detection, characterization and imaging of materials and objects in a dense compressive media comprising:
(a) a means for exciting regions, materials and objects in a dense compressive media employing a plurality of ultrasound wave beams in combination with
(b) a microwave means for detecting, characterizing and imaging the excited materials and objects.

2. A method for detection, characterization and imaging of materials and objects in a dense compressive media comprising the steps of:
(a) exciting regions, materials and objects in a dense compressive media by transmitting a plurality of ultrasound wave beams into the region; and
(b) detecting, characterizing and imaging the excited materials and objects employing microwave means.

3. A system for detection, characterization and imaging of materials and objects in a dense compressive media comprising:
(a) a means for generating input microwaves;
(b) a means for transmitting said input microwaves into the dense compressive media;
(c) a means for generating a plurality of input ultrasound waves having small differential frequencies;
(d) a means for transmitting said plurality of ultrasound waves into the dense compressive media;
(e) a means for detecting microwaves reflected by boundaries, materials and objects in the dense compressive media;
(f) a means for processing detected microwaves into information describing the presence, location and characteristics of the materials and objects; and
(g) a means for displaying said information.

4. The system of claim 3, further comprising:
(a) a means for generating an additional ultrasound wave for detecting motion induced by the plurality of input ultrasound waves;
(b) a means for transmitting said detection ultrasound wave into the dense compressive media;
(c) a means for detecting ultrasound waves reflected by the materials and objects excited by the multiple input ultrasound waves;
(d) a means for processing the detected ultrasound waves into information describing the presence, location and characteristics of the excited materials and objects; and
(e) a means for displaying said information.

5. The system of claim 3, further comprising:
(a) a hydrophone for detecting acoustic waves generated by the excitation of materials and objects;
(b) a means for converting the detected acoustic waves into information describing the presence, location and characteristics of the excited materials and objects; and
(c) a means for displaying said information.

6. A system for detection, characterization and imaging of materials and objects in a dense compressive media comprising:
(a) an ultrasound subsystem for generating a plurality of ultrasound waves for exciting materials and objects in the dense compressive media; and
(b) a microwave imaging subsystem for detecting, characterizing and imaging said excited materials and tissues.

7. The system of claim 6, further comprising:
(a) a means for generating an additional ultrasound wave for detecting motion induced by the plurality of input ultrasound waves;
(b) a means for transmitting said detection ultrasound wave into the dense compressive media;
(c) a means for detecting ultrasound waves reflected by the materials and objects excited by the multiple input ultrasound waves;
(d) a means for processing the detected ultrasound waves into information describing the presence, location and characteristics of the excited materials and objects; and
(e) a means for displaying said information.

8. The system of claim 6, further comprising:
(a) a hydrophone for detecting acoustic waves generated by the excitation of materials and objects;
(b) a means for converting the detected acoustic waves into information describing the presence, location and characteristics of the excited materials and objects; and
(c) a means for displaying said information.

9. A method for detection, characterization and imaging of materials and objects in a dense compressive media comprising the steps of:
(a) generating input microwaves;
(b) transmitting said microwaves into the dense compressive media;
(d) generating a plurality of input ultrasound waves;
(e) transmitting said input ultrasound waves, and the resultant low-frequency, high displacement force beat frequency ultrasound waves, into the dense compressive media to excite materials and objects in the media;
(f) detecting microwaves reflected by the excited materials and objects;
(g) converting said detected microwaves into information describing the presence, location and characteristics of the excited materials and objects; and
(h) displaying said information.

10. A system for detection, characterization and imaging of materials and objects in a dense compressive media comprising:
(a) an ultrasound subsystem further comprising a plurality of waveform generators to produce ultrasound waveforms of differential frequency, a plurality of power amplifiers to condition the generated ultrasound waveforms, a plurality of ultrasound transducers to transmit the conditioned ultrasound wave into the target media and excite materials and objects within the media, and a scan controller/actuator to enable scanning of the media; and
(b) a microwave imaging subsystem further comprising a microwave generator for producing microwaves, a power amplifier to condition the generated microwaves, a microwave antenna or antenna array to transmit the conditioned microwave into the target media and to detect microwaves reflected by media boundaries and materials within the media, a computer/signal and data processor to process detected analog microwave signals into information describing the presence, location and characteristics of the excited materials and objects, and a display for communicating the information.

11. The system of claim 10 wherein the plurality of ultrasound transducers is embodied in an ultrasound transducer array.

11. The system of claim 10 wherein the plurality of ultrasound transducers is embodied in a confocal ultrasound transducer array.

12. The system of claim 10, further comprising an additional ultrasound transducer for detecting ultrasound waves reflected by materials and objects within the media.

13. The system of claim 10, further comprising a hydrophone for detecting acoustic waves generated by the motion induced in materials and objects within the media.

14. The system of claim 10 wherein a single ultrasound transducer transmits the plurality of ultrasound waves into the media.
